(19) United States
(12) Patent Application Publication
(10) Pub. No.: US 2009/0038069 A1
(43) Pub. Date: Feb. 12, 2009

(54) HYDRO-THERAPEUTIC MASSAGE DEVICE

(76) Inventor: David Heilman, Geneseo, IL (US)

Correspondence Address: HAMILTON IP LAW, PC, 331 W. 3RD ST., NEW VENTURES CENTER SUITE 120, DAVENPORT, IA, 52801, US

(21) Appl. No.: 12/017,329

(22) Filed: Jan. 21, 2008

Related U.S. Application Data
(63) Continuation-in-part of application No. 11/980,940, filed on Oct. 31, 2007.
Application No. 11/980,940 is a continuation of application No. 11/046,016, filed on Jan. 31, 2005, now Pat. No. 7,320,438.
(60) Provisional application No. 60/551,522, filed on Mar. 5, 2004.

(51) Int. Cl. A61H 33/00; A61F 5/00

(52) U.S. Cl. 4/541.6; 600/38

(57) ABSTRACT

Method and apparatus for hydrotherapeutic stimulation having a handle with housing with exterior engagement surfaces for contacting a user's lower body. An air-water stream generator, coupled to a water inlet and an air inlet, positioned in housing and a pump means imparts an increase in pressure and flow of water within housing to outlet nozzle. An electrical source is electrically connected to the pump means within the housing. An air-water stream outlet nozzle located within the fore portion of the housing and connected to air-water stream generator for transmission of air-water stream onto user's lower body positioned fore of air-water stream outlet nozzle. Either distended portion or attachment at fore portion of housing, upon insertion in an anal or a vaginal orifice in the user fixes the position of housing in relation to user's lower body thereby allowing handle further control of air-water stream outlet nozzle and stimulation.

### CROSS REFERENCE TO RELATED APPLICATIONS

I, David Heilman, residing in Geneseo, Ill. and being a United States citizen, do herein file this non-provisional continuation-in-part patent application entitled “Hydro-therapeutic Massage Device” and claim priority from non-provisional U.S. patent application assigned application Ser. No. 11,980,940 filed on Oct. 31, 2007 entitled “Feminine Hydro-therapeutic Massage Device”, which was a continuation of non-provisional U.S. patent application assigned Ser. No. 11,046,016 filed on Dec. 18, 2004, which claimed priority from provisional U.S. patent application No. 60,551,522 filed on Mar. 5, 2004, all of which are incorporated by reference herein.

### FIELD OF THE INVENTION

This invention relates to the field of portable hydro-therapeutic stimulation devices for massage therapy to a user's genital region wherein a method of circulating the water within a small contained body of water such as a spa, shallow pool, or a bathtub is utilized to produce the desired effect.

### STATEMENT REGARDING FEDERALLY SPONSORED RESEARCH OR DEVELOPMENT

No federal funds were used to develop or create the invention disclosed and described in the patent application.

### REFERENCE TO SEQUENCE LISTING, A TABLE, OR A COMPUTER PROGRAM LISTING COMPACT DISK APPENDIX

Not Applicable

### AUTHORIZATION PURSUANT TO 37 C.F.R. §1.171 (d)(c)

A portion of the disclosure of this patent document contains material which is subject to copyright and trademark protection. The copyright owner has no objection to the facsimile reproduction by anyone of the patent document or the patent disclosure, as it appears in the Patent and Trademark Office patent file or records, but otherwise reserves all copyrights whatsoever.

### BACKGROUND OF THE INVENTION

A wide variety of mechanical stimulating devices and a limited number of hydro-therapeutic devices are known in the prior art to affect a massage to the human body to promote stress relief and relaxation. It is a known fact to those skilled in the art that direct stimulation of the typical female clitoris will result in a climax of the entire body, and a plethora of devices have been developed to this end. Mechanical vibratory stimulating devices are diverse and can be used to massage all parts of the human body to effect a soothing relaxation of local musculature and may be used to focus a concentrated effect on a female user's clitoral stimulation point. A general whole body therapy is achieved by use of spas and whirlpools where a plurality of hydro jets are placed around the periphery of the user in a water filled tub setting allowing for circulation, heating and aeration of the water. Hydro pulsating devices in fluid connection with an external water source or a circulated water source are employed in showers, tubs, whirlpools, and personal spas to affect a similar but deeper massage than the vibratory devices. A few of these personal spas employ a method of a hand held local massage as an option.

More recently personal spas and whirlpools have evolved in shape and design to provide a contoured comfortable seating arrangement with strategically placed hydro jets to provide stimulation to predetermined points of the human anatomy such as the back, neck, legs, feet, and pelvic region.

U.S. Pat. No. 5,920,923 issued to Jillette teaches hydro clitoral stimulation in combination with a fixed whirlpool spa environment. As taught by Jillette, and similar to the present invention, air entrained in water allows stimulation to the female genitalia. The unit taught by Jillette, however, is not portable and would be quite costly to purchase, install and maintain. Furthermore, Jillette does not teach a method or apparatus allowing fixed placement combined with fine control of the air-water stream during stimulation.

U.S. Pat. No. 5,077,841 issued to Sugai employs a portable self-contained pumping unit designed to circulate water within a bathtub. The discharge of the pumping unit utilizes a hydro jet with an entrained air mix that is directed at target points to affect a local massage as the user positions oneself near the discharge of the device. A rechargeable battery is used to power the pumping unit. Multiple discharge paths are used to allow the user to select the type of water stream exiting the device. The housing is temporarily attached to the wall of the tub.

U.S. Pat. No. 4,924,535 issued to Yamasaki is based on the same principle as the previously discussed Sugai patent, and uses an external power supply. Yamasaki requires a fixed mounting that would not be easily positioned within the bathtub and allows only limited adjustment by the user. Yamasaki is not practical to effect the variable local stimulation sought by the user. Additionally, Yamasaki provides no teaching on direct or indirect control methods. Furthermore, Yamasaki does not teach any engagement surfaces allowing placement between the user's legs.

U.S. Pat. No. 4,123,808 issued to Guarrera teaches a general area cleansing system using water without aeration. To employ the system, the user most permanently mount a chair to the bathtub. Furthermore, during cleansing, the user has no contact with the nozzle or structure directing the water to the cleansing area thus, limiting the user's control of the air-water stream upon the user.

U.S. Pat. No. 3,902,200 issued to Pratt also teaches a general area cleansing system, referred to as a “Sitz” bath, which also uses water without aeration. To use the system as taught by Pratt, the user must sit immobile in a curved chair located in a bathtub. Pratt does not teach a method or apparatus for adjustment of the direction of the water used for cleansing. Pratt does not teach a method or apparatus allowing engagement between the user's legs or comfortable access to the frontal areas of the user.

U.S. Pat. No. 4,100,917 issued to Talge et. al (“Talge”) teaches a portable water circulating device that mounts over the side of the tub to supply a water air mixture over a general area on the bottom of the tub. An optional handheld device connected by a hose to the unit capable of reaching more specific target areas is also taught. No means for removable fixation of the optional handheld device to the tub walls is taught by Talge.

U.S. Pat. No. 5,807,289 is a portable hand held hydro massage device for submerged use and focused stimulation to a user's body parts. As disclosed, this patent does not teach an apparatus or method allowing fixed use of the device.

The prior art disclosed and discussed above indicates weaknesses in the teachings necessary for hydro-therapeutic stimulation of the female genitalia and surrounding regions. The prior art fails to teach finite control of the stimulation stream. The prior art fails to teach a device or method allowing the user to be both comfortable and stimulated. The prior art fails to teach a device or method that allows control of the stimulation stream through engagement between the device and the user's lower body. The prior art fails to teach a protrusion or attachment which may be inserted in the user during use to fix the relationship between the user and the hydro jet device. The prior art also fails to teach a submergible device rechargeable device capable of generating a stimulation stream. The common thread linking the failure of the prior art is the lack of individual control and choice of control for the user. This lack of teaching, to date, thus limits the effectiveness of using water for stimulation of the user.

### SUMMARY OF THE INVENTION

The present art as described herein overcomes the deficiencies of the prior art. The apparatus disclosed and described herein addresses the weaknesses found in the prior art and improves hydrotherapy stimulation devices by providing a small mobile device capable of deployment within any shallow contained bathing area such as a spa, pool or bathtub. In one embodiment of the device, it is fixable, although still mobile, to the water container walls. The combination of removability and fixability allows the user to find a comfortable spatial relationship between the device, the user and the user's position in the contained bathing area, and then maintain that position. In another embodiment of the device, the spatial relationship between the user and the device may be fixed by either a protrusion extending from the device, wherein the protrusion may be inserted in the user or by connecting a detachable attachment to the device, which may be inserted in the user. After establishing and fixing that relationship, the user may use the side engagement surfaces of the device to position the apparatus between the user's legs at a high comfort level, as determined by the user. The user may then also control directly at a fine level the water stream and thus the area of treatment during stimulation. Because treatment and stimulation are a dynamic process, the direct and indirect control surfaces allow the user to find the best stimulation area and rate of stimulation for the particular user, at a particular time.

A portable pumping unit contained in a housing designed to receive the female pelvic region and direct a focused aerated pulse of water at the same. Said portable hydro massage device when submerged in an enclosed body of water containing a female user, such as a bathtub or pool, provides a local massage to the labium region and a focused stimulant to the female user's clitoris thereby resulting in a superior sensation to the entire body promoting an overall euphoric state and calming effect. The apparatus employs a saddle shaped housing, where the buttocks are placed, and a handle for controlling the concentration of the stimulus. Disposed within the midsection of the handle is a hydro jet with a driven flow from a circulating water pump that draws suction from the main body of water. The hydro jet is adjustable in pressure and a method is provided for aeration of the water discharged to allow for a rhythmic pulsation effect. The handle may be held and the lower body positioned as needed to focus the discharged water from the hydro jet to the desired point of the pelvic region.

The climatic effects are superior to the typical mechanical vibration devices and the prior hydro-therapeutic devices previously described. When operating, the female user has control of the unit to vary the angle and intensity of the stream and alternately sit or recline in a rested position with the legs extended or drawn up near the body. The device is portable and can be moved to different locations. In one embodiment, a removably secure base, fixed to the bottom of the tub, is required to allow for a moderate amount of pressure from the user's pelvic to be applied to the unit. In another embodiment, the spatial relationship between the user and the hydro jet device is generally fixed by a protrusion from the device or an attachment, either of which may be inserted into an orifice of the user.

It is therefore an objective of the invention to provide a hydrotherapeutic and stimulation method and device that allows fixable attachment within a self-contained bathing area.

It is another object of the invention to provide comfortable access between the user's legs without direct engagement of the apparatus with the user's vaginal region.

It is another objective of the invention to provide a utility for local concentrated stimulus to the female user's labium region that is socially acceptable and does not require penetration of the vaginal opening to achieve the desired effect.

It is another objective of the invention to combine close proximity of the discharge nozzle to the labium area with an adjustable discharge stream trajectory for user stimulation and “hands-free” adjustment. The inventor has realized the need for fine adjustments of the discharge nozzle at critical moments and mental concentration would be broken as the hand blocks the discharge stream or the device has to be moved away from the body to make adjustments.

It is another objective of the present invention to allow indirect adjustment of the discharge stream trajectory with minimal change of the discharge stream intensity by moving the housing of which the discharge nozzle is mounted.

It is another objective of the invention to provide a hydrotherapeutic and stimulation method and device that allows the relationship between the user and the device to be fixed within a self-contained bathing area by use of an attachment that is inserted in the user.

### BRIEF DESCRIPTION OF DRAWINGS

FIG. 1 is a side elevational view of the invention in a typical application. This first embodiment is shown in a sectional side view of a typical bathtub. The control unit is displayed attached to the outside wall of the bathtub.

FIG. 2 is a top plan view of the first embodiment.

FIG. 3 is a rear elevational view of the first embodiment.

FIG. 4 is a right side elevational view of the first embodiment depicting the range of movement of the device.

FIG. 5 is a right side elevational view of the first embodiment with a cutaway section of the housing to show the internal configuration of internal parts.

FIG. 6 is a perspective view of pivotal slide assembly.

FIG. 7 is a cross sectional view of pivotal slide assembly.

FIG. 8 is a rear elevational view of another embodiment.

FIG. 9 is a side elevational view of the embodiment shown in FIG. 8.

FIG. 10 shows a partial cross sectional view of the preferred embodiment slide section with an extending rotatable pivot point.

FIG. 11 shows a typical bumper and method of attachment.

FIGS. 12A and 12B shows an optional bumper that maintains clearance from the user in relation to the discharge of the hydro jet.

FIGS. 13A and 13B shows an optional bumper that maintains clearance from the user in relation to the discharge of the hydro jet and splits the discharge flow into two separate streams.

FIG. 14A shows a partial front elevation view of the handle.

FIG. 14B shows a partial side sectional view of the handle.

FIG. 14C shows a side elevation view of an extended pivotal bumper and attachment method.

FIG. 14D shows a side elevation view of a stationary bumper and attachment method.

FIG. 14E shows a top view of stationary bumper and attachment method.

FIG. 15A shows a front elevation view of the preferred embodiment.

FIG. 15B shows a side elevation sectional view of non-electric preferred embodiment.

FIG. 15C shows a side elevation view of the preferred embodiment in an alternate position.

FIG. 16 shows a side cross section of another portable embodiment with additional features including a rechargeable battery.

FIG. 17 is a graphical representation of the product in use by a female user.

DETAILED DESCRIPTION - LISTING OF ELEMENTS

| ELEMENT DESCRIPTION | ELEMENT # |
|---|---|
| Housing | 1 |
| Handle | 2 |
| Feet | 3 |
| Pumping unit | 4 |
| Electrical conductors | 5 |
| Suction inlet (water) | 6 |
| Discharge | 7 |
| Hydro jet | 8 |
| Water inlet port | 9 |
| Suction conduit (air) | 10 |
| Hydro jet air inlet | 11 |
| Discharge nozzle (orifice) | 12 |
| Body of water | 13 |
| Bathtub | 14 |
| Power Supply | 15 |
| Switchable control | 16 |
| Bumper | 17 |
| Fluid conduit | 18 |
| Hydro jet inlet | 19 |
| Air control regulating valve | 20 |
| Pivotal slide mechanism | 21 |
| Pivotal point | 22 |
| Second position | 23 |
| Third position | 24 |
| Overlapping seam | 25 |
| Male slide section | 26 |
| Male pivot part | 27 |
| Releasable stop tab | 28 |
| Aperture | 29 |
| Female slide section | 30 |
| Ball and socket pivot point | 31 |
| Suction cup 32 | 32 |
| Male slide section 33 | 33 |
| Stop collar | 34 |
| Stop tab | 35 |
| Base | 36 |
| Female slide section 37 | 37 |
| Aperture 38 | 38 |
| Male part 39 | 39 |
| Relief cutaway portion 40 | 40 |
| Nodal protrusion | 41 |
| Female ball sockets | 42 |
| Attachments | 43 |
| Male portion of ball joint | 44 |
| Inlay | 45 |
| Suction cup | 46 |
| Air-water stream | 47 |
| Supply hose | 48 |
| Chamfered portion | 49 |
| Bottom tub wall | 50 |
| Vent hole | 51 |
| Ridges | 52 |
| Sealed contacts | 53 |
| Battery lead wires | 54 |
| Hydro massage device | 55 |
| Diodes | 56 |
| Jet nozzle | 57 |
| Upper labium and clitoral region | 58 |
| Rechargeable battery pack | 59 |
| Motor unit | 60 |
| Pumping section | 61 |
| Waterproof switch | 62 |
| Oscillating jet pattern | 63 |

### DETAILED DESCRIPTION

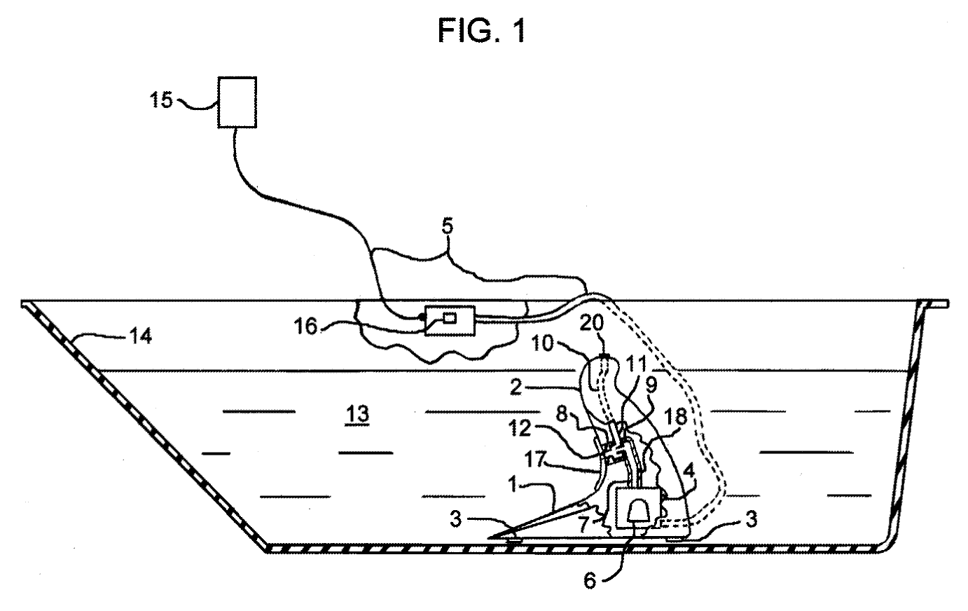

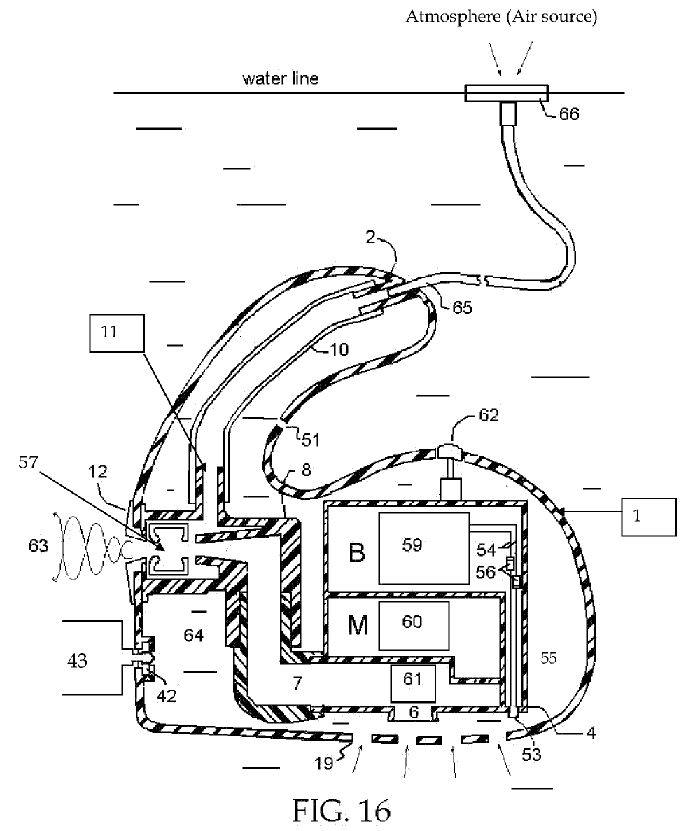

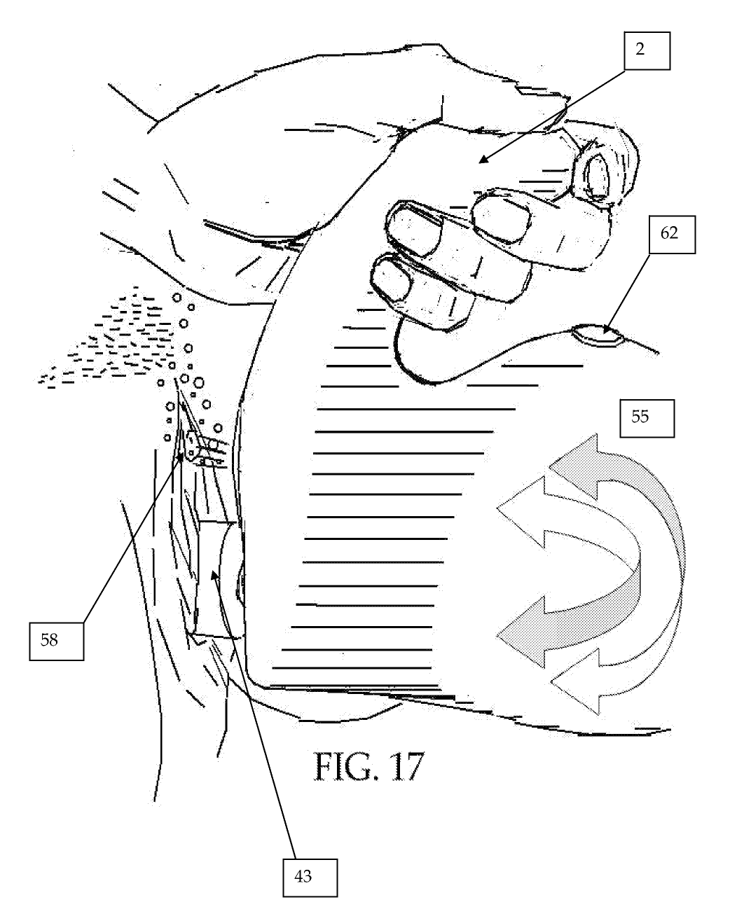

FIGS. 1-15 illustrate a first embodiment of hydro massage device 55. Referring to FIG. 1, the hydro massage device 55 is functional when submerged in a main body of water 13, e.g. contained in a standard bathtub 14. The hydro massage device 55 consists of a housing 1, which functions as a partial seat and floodable housing for the internal parts and provides support for a variably disposed elongated protrusion from the forefront of the housing 1 heretofore referred to as the handle 2. The handle 2 allows the user to hold and control the direction of the hydro massage device 55 and may be formed in any number of shapes that would be of a comfortable grip to the user. In the embodiment shown, by way of illustration and without limitation, housing 1 is formed with a contour concave taper, sloping gradually from the rear of the housing 1 up to the fore of the housing 1 at the base of the handle 2 to aid in positioning the user's pelvic region into proximity of the handle 2 and to act as a base for anchoring the hydro massage device 55 to the bottom of tub 14 using the weight of the user. The housing 1 may employ slip resistant feet 3 to prevent the hydro massage device 55 from shifting foreword as the user mounts upon or in close proximity to said device. Furthermore, the housing 1 contains a circulating water pumping unit 4, and electrical conductors 5 to feed low voltage direct current power from a conventional external AC to DC power supply 15 to said pumping unit 4. A switchable control 16 connected in the electrical power supply conductors 5 enables the user to turn on or off said pumping unit 4. In another embodiment, by way of illustration and without limitation, and as illustrated in FIGS. 16-17, a waterproof electrical switch 62 may be employed within the hydro massage device 55 to allow the user to switch off and on the hydro massage device 55 and a sealed rechargeable battery pack 59 may be employed within the housing 1 to provide the electricity needed to operate said pumping unit 4.

Referring to FIG. 1 and FIG. 5, said pumping unit 4 consists of a direct current electrical motor coupled to a centrifugal pump capable of running submerged in water and is common to those skilled in the art. Said pumping unit 4 has a suction inlet 6, which draws water, flooded within the housing, and discharges the water at a higher pressure and flow from the discharge 7 of said pumping unit 4. Disposed within said handle 2 is hydro jet 8, an air-water structure means common to those skilled in the art, externally discharging through discharge nozzle or orifice 12 toward the user from the mid-section of the handle 2. A water inlet port 9 of said hydro jet 8 is in fluid communication through fluid conduit 18 extended to the discharge 7 of said pumping unit 4. Said hydro jet 8 increases the velocity of the water through a venturi effect to draw in air supplied by air suction conduit 10, connected at hydro jet air inlet 11, further extended above the surface of the water at the top of said handle 2 to provide aeration to discharge nozzle 12 of the hydro jet 8. An air control regulating valve 20, common to those skilled in the art, is employed in communication with said air suction conduit 10 to allow for variable airflow into the hydro jet 8 and ultimately the intensity of the water discharged through nozzle 12 of said hydro jet 8. Air suction conduit 10 alternately may be positioned at any other desired point on the housing 1 that provides for contact with an air source. As shown in FIG. 17, by way of illustration and without limitation, the hydro jet 8 is oriented such that the discharge nozzle 12 delivers an air-water stream 47 towards the female user's upper labium and clitoral region(s) 58 when the female user is positioned in front of the housing 1. It will be apparent to those of ordinary skill, without limitation and by way of illustration, that the hydro massage device 55 may be rotated to direct the air-water stream 47 to the anus. Although the present art is illustrated with a female user, it is without limitation and has application to male users and as such is not limited by FIG. 17.

Referring to FIG. 4 and FIG. 5, the vertical placement and orientation of said hydro jet 8 in relation to said housing 1 is designed to accommodate a wide variety of users. A female user may shift her pelvic region and sit or recline while positioned adjacent the housing 1 and direct the massaging discharge of the hydro jet 8 to the desired point of the labium region by moving the handle 2 with one or both hands. To utilize the handle 2 function to make adjustable the focus of the hydro jet 8 discharge trajectory, as referenced by trajectory lines A, B, and C, a pivotal point 22 is employed by a pivotal slide mechanism 21. An overlapping seam 25 is utilized to separate said the housing 1 from said handle 2. The internal lower extents of the handle 2 are pivotally connected at said pivot point 22 and in turn coupled to said slide mechanism 21. Said pivot point 22 and said slide mechanism 21, in conjunction with overlapping seam 25, allow said handle 2 to be moved vertically and or pivotally fore and back in relation to housing 1. Said housing 1 internally employs a recessed female socket whereas male portion of slide mechanism 21 is movably received to allow vertical movement of pivotally coupled said handle 2. Reference line A shows the trajectory of the discharge of said hydro jet 8 with said handle 2 at a first position. Reference line B shows trajectory of the discharge of said hydro jet 8 with said handle 2 pivoted foreword to a second position 23. Reference line C shows trajectory of discharge of said hydro jet 8 with said handle 2 extended vertically from second position 23 to a third position 24. The discharge trajectory from the hydro jet 8 is then variable, at the users discretion, as said handle 2 may be moved to any position within the range of the first position, said second position 23 and said third position 24. Vertical movement of said handle 2 to said third position 24 is limited by releasable stop tab employed in slide mechanism 21.

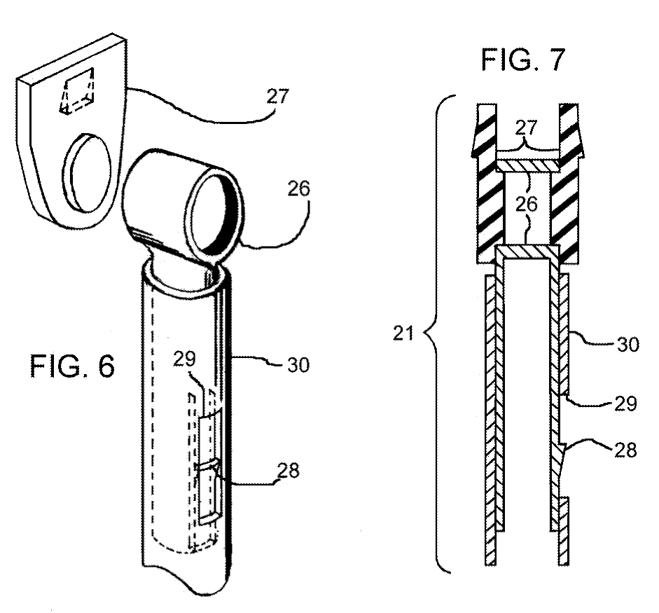

Referring to FIG. 6 and FIG. 7, releasable stop tab 28, engaged in aperture 29 of female slide section 30, is flexibly employed on male slide section 26 of said slide mechanism 21 to limit vertical movement which prevents slide mechanism 21 from disengaging during normal use but is releasable to allow slide mechanism 21 to be separated for device disassembly. Two male pivot parts 27, one not shown in FIG. 6, attach at the base of said handle 2, engage both side ends of female pivot part of said male slide section 26 to allow pivotal movement of attached handle 2. Female slide section 30 is molded into base of said housing 1. Two male pivot parts 27 can be molded into base of said handle 2. Slide mechanism 21 may employ a ball and socket pivot point to perform the same function as described above additionally providing for a rotatably swiveled pivot point and a greater range of trajectory paths from the hydro jet discharge.

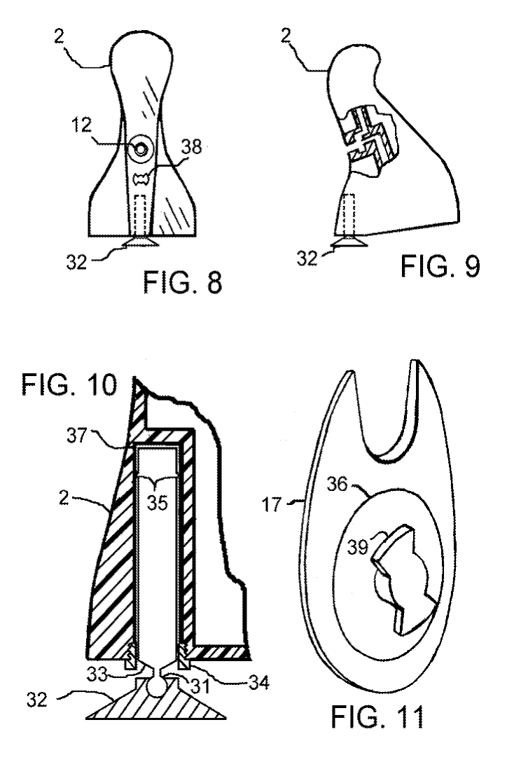

Referring to FIG. 8, FIG. 9, and FIG. 10, another embodiment is depicted allowing an user to affix the device directly to the bottom of the tub, between the legs, and adjust movement of said handle 2 as desired. This embodiment is functionally identical to the first, and employs the same mechanical and electrical parts to operate. The slide mechanism, however, is modified to allow said handle 2 to be used independent from housing 1. This embodiment employs a ball and socket pivot point 31 at the base of handle 2, providing for an extending rotatably swiveled pivot point. Suction cup 32 receives ball end of male slide section 33 allowing for a removably fixed attachment to the bottom of tub. Female slide section 37 is molded into base of handle 2 to receive said male slide section 33 allowing said handle 2 to be moved pivotally within the physical range of ball and socket pivot point 31. A stop tab 35 is molded into male slide section 33 which will impinge on stop collar 34 to prevent the slide assembly from disengaging when extended to its physical range of motion.

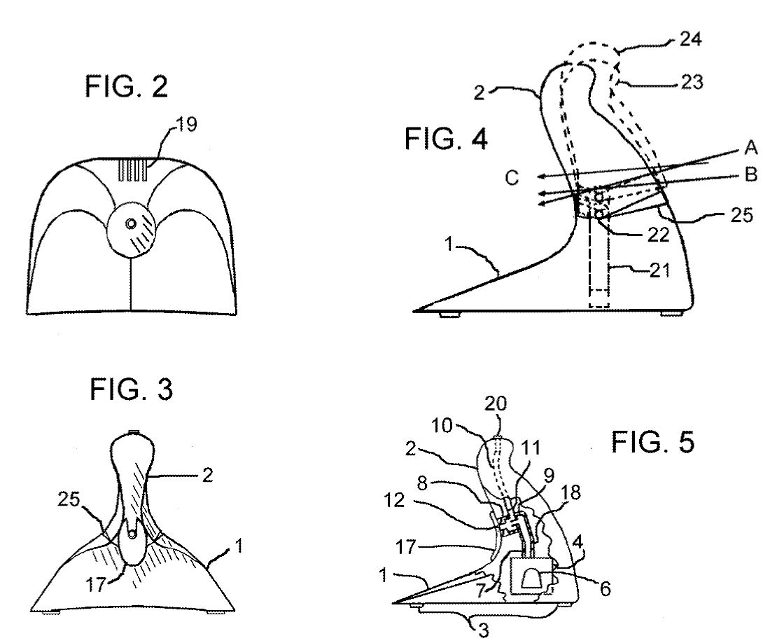

Referring to FIG. 2, the floodable housing is shown in the top view with slots 19 which communicate the surrounding water to the interior of the hydro massage device 55. It may be desired to include a filter assembly (not shown) at slots 19 to prevent debris from entering the pumping unit 4. Apertures, functionally identical to said slots 19, may be utilized beneath the housing to allow water to be pulled in and flood the internal cavity from the bottom of the housing 1.

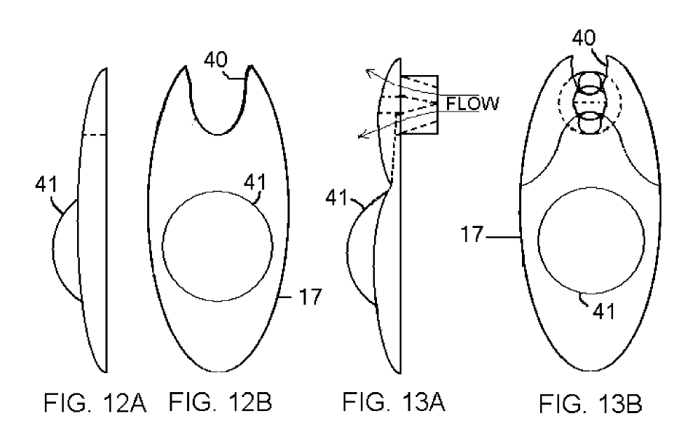

Referring to FIG. 3, FIG. 12, and FIG. 13, externally surrounding the outlet of the hydro jet 8 is a bumper 17 allowing for comfortable pressing of the user's pelvic region to said handle 2 as desired. Said bumper 17 is attached to handle 2 and not to housing 1 thereby extending over said seam 25. As said handle 2 is moved to a variable position, said seam 25 will not be exposed to the user's labium region. Bumper 17 can be designed to enhance the user's experience and function to maintain a small distance between the user and the exterior edge of the discharge of hydro jet 8 to prevent blocking of the discharge flow. Bumper 17 is employed in all embodiments to perform a similar function.

Referring to FIG. 12 and FIG. 13, two different embodiments of said bumper 17 are depicted, respectively. In both embodiments, as shown in FIGS. 12B and 13B, said bumper 17 has a relief cutaway portion 40 at the top of said bumper 17 and a thickness to maintain a path for discharge flow from the hydro jet 8 if the user presses the labium region hard against bumper 17. A nodal protrusion 41 is employed to stimulate the vaginal opening and can be of various shapes and lengths. The embodiment of the bumper 17 shown in FIGS. 13A and 13B, is configured such that the single discharge flow from the hydro jet 8 is split into two channel streams, one directed up and the second down, to provide further stimulation through water contact at two distinct points. Although not shown, those practiced in the arts will understand that it is also contemplated by the inventor to modify bumper 17 with other variations of cutaway portion 40 to include a plurality of channel streams emitting from said hydro jet massage device 50.

Referring to FIG. 8 and FIG. 11, said bumper 17 is removeably attached to the rear face of said handle 2 immediately below hydro jet discharge nozzle 12 by means of a bow tie shaped aperture 38. Aperture 38 receives a corresponding male part 39 fixed to the base 36 of bumper 17. Male part 39 is locked into aperture 38 by inserting, then twisting 90 degrees to lock the bumper base into place. This feature allows a variety of bumper shapes to be interchanged as desired by the user or removed for cleaning or replacement.

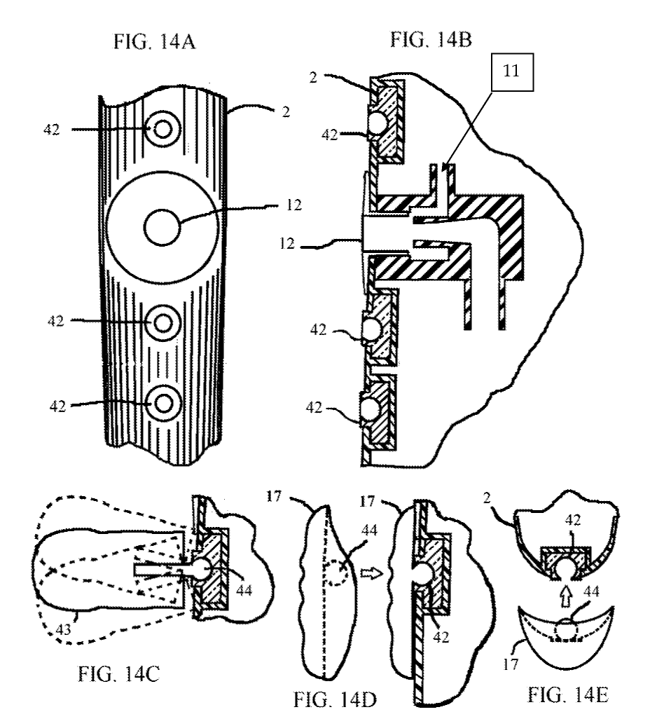

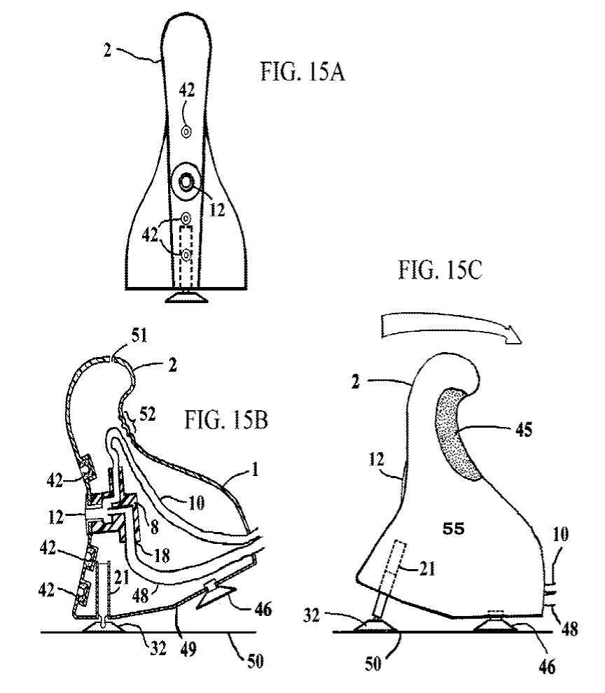

As shown and illustrated, without limitation, in FIGS. 14A-E, another method of bumper attachment to handle 2 is utilized with ball and socket joints. Female ball sockets 42, made of rubber, are disposed above and or below the hydro jet discharge nozzle 12 to allow a pivotal attachment, as in 14C, or a stationary attachment of bumper 17 shown in FIGS. 14D, 14E to allow the user to easily attach and remove for interchange, and connect attachments 43 to any available socket. Male portion 44 of ball joint, made of plastic, is molded into fixed bumper 17 or a variably disposed phallus shaped attachment 43. With this method of attachment, said phallus shaped attachment 43 is pivotal at the attached end and will allow for angular changes as the user shifts position of said handle 2. A wide variety of bumper 17 or attachment 43 shapes, both stationary and pivotal, can be designed and made to connect to the housing 1 with this method. By way of illustration and without illustration, attachment 43 may be formed as a phallic extension with a g-spot massager (not shown) or as a prostrate massager (not shown). Phallic extensions with these particular functions are known in the art so no further discussion is necessary.

FIGS. 15A-C depict an alternative embodiment of the present invention employing an externally supplied water source via hose 48 instead of an electric pump. Supply hose 48, connected to water inlet port 9 of said hydro jet 8 is in fluid communication with a bathtub waterspout or a jet nozzle of a hot tub, or any other water pressure source. The suction conduit 10 may be routed within the housing 1 and exit at any penetration point on the housing and extend to the air space above the water line to maintain contact with an air source. Hydro jet 8 discharges the mixture of air and water at discharge nozzle 12 when water under pressure is supplied through hose 48 and air is venturied in through air suction conduit 10. This means eliminates the need for an electric power source and pump with no loss of function.

In FIG. 15B the base of the unit has a chamfered portion 49 positioned on the lower side of housing 1 to such a degree that the unit can be tilted back, FIG. 15C, with the slide extension 21, fixed and extended, and optional suction cup 46 making a fixed second point of contact. This feature removeably fixes the device in a stationary position and allows hands free operation during stimulation.

In FIG. 15C, the upper extents of the handle 2 can be coated, textured, or an inlay 45 made of a rubber compound to make a non-slip, comfortable grip for the user. A series of ridges 52, FIG. 15B, may also be employed at the back or front of neck on said handle 2 to provide a gripping surface. Said handle 2 is slender and a plurality of shapes may be designed to fit comfortably in the hand of the average female user.

In FIG. 15B, vent hole 51 is utilized to continuously remove air from the internal volume as housing 1 floods with water from the bottom and the air is displaced. Said vent hole 51 is located at the top of said handle 2 to minimize buoyancy of the device.

In a typical session the female user places said housing 1 into a tub of water drawn to the desired temperature to suit the bather. When the embodiment shown in FIGS. 1-14 is used, said switchable unit 16 is placed on the outside edge of the tub and the electrical supply 15 is plugged into the switchable unit 16. The female user may now enter the bath and position said housing 1 to a desired distance from the back of said tub 14 and take a seated position proximate said housing 1. The user may now turn on the power to the device at said switchable unit 16 and adjust the seating position in the housing 1 or recline her upper torso against the rear wall of the tub 14. The user's legs may be extended without interference or may be drawn up in a splayed position. Placing a hand on said handle 2 as leverage, the female user now may position her pelvic region at the desired distance from or up to said bumper 17. The user may adjust the velocity of the air-water mixture discharging from hydro jet 8 by adjusting said air control valve 20 to the desired stimulation intensity. Said handle 2 may be moved to a plurality of positions to fine tune the trajectory of the discharge stream to focus the discharge at the desired stimulation point(s).

The materials required to construct the unit could be of injection molded or blow molded plastic. The bumper can be made of silicone, soft plastic or rubber compound. The pumping unit is common to those skilled in the art as well as the hydro jet and all required tubing and electrical parts to construct the hydro massage device 55.

Referring to FIG. 16, a cordless portable battery operated embodiment of the present invention is depicted. Previously described pumping unit 4 is further defined by rechargeable direct current battery pack 59, motor unit 60, and a pumping section 61. Pumping section 61 is driven by motor unit 60 to produce suction at pump inlet 6 and establish a flow rate of water at pump outlet 7. Motor unit 60 is electrically connected, but intrinsically separated from rechargeable battery pack 59 and both are contained within sealed, waterproof compartments, M and B respective, to withstand submersion depths to 1 meter, without limitation. The battery is electrically recharged on a charging cradle (not shown) which makes contact with a set of sealed contacts 53 which terminate to the battery with wire leads inside the sealed battery compartment B. The battery lead wires 54, electrically connect to sealed contacts 53, and are protected by diodes 56 so no voltage potential or current flow exists at sealed contacts 53 when the hydro massage device 55 is in use.

In another embodiment, a contact-less battery charger can be employed to eliminate external charge contacts 53. This type of charger is common to those skilled in the art and is often used for devices intended for use in wet locations. Electrical charge flux is transferred across an air gap between a secondary pickup coil within device and a primary charging coil embedded within the charge cradle.

On-Off power is controlled by the user with sealed waterproof switch 62. Circuitry may be employed to automatically shut off the device when battery voltage drops to a predetermined level.

Referring to FIG. 16, a jet nozzle 57 with feedback loops, common to those skilled in the art, is utilized to form an optional embodiment of hydro jet 8 that can be employed to affect an oscillating jet pattern 63 from nozzle 12. See U.S. Pat. No. 4,905,909 issued to Woods for “Fluidic Oscillating Nozzle”, which is incorporated by reference herein, for further background and discussion on oscillating jet nozzles. The oscillating jet pattern 63 produces a rapid side-to-side flutter effect to the target surface of the user and provides for an increased stimulus with or without air intake mixed into the water stream. Utilizing this method, nozzle 12 can be designed to rotate ninety (90) degrees to allow the user to select a side to side or up and down flutter pattern.

Another embodiment of the present invention is illustrated, without limitation, at FIG. 16. As illustrated, air suction conduit 10 terminates at the top of handle 2 with a resulting vent hole 51 or penetration in the housing 1 at the top of the handle 2. When handle 2 is held by the user, the user may use their finger or thumb to throttle the air suction conduit 10 for power output adjustment or pulse the air suction conduit 10 by a rapid tapping motion over the vent hole 51 to affect a pulsed output from the hydro massage device 55.

Air suction conduit 10 extends to the top of handle 2. If used in very deep water, submersion of the hydro massage device 55 (and air suction conduit 10) effectively terminates air intake as water covers vent 51. To maintain air intake in deeper water, an extension tube 65 which plugs into air suction conduit 10 and extends to the surface of the water to freely communicate air to the hydro jet air inlet 11 is disclosed and claimed herein. (See FIG. 16) Floating bobber 66 is employed at the end of tube 65 to maintain air intake to the hydro massage device 55 by floating at the water line with no restraints or mounts. In an optional embodiment, the floating bobber 66 is constructed to function as an air pulsation device (not shown), by which means the air intake path is alternately opened and closed to effect a pulsation at the outlet nozzle 12.

The embodiment in FIG. 16 eliminates the supporting mount affixed to the bottom of the tub. In this manner, the device is unsecured to a support surface allowing further positioning, as necessary. If the user employs an attachment 43, in place of bumper 17, having a phallic shape, with subsequent insertion into the vaginal canal, as shown without limitation at FIG. 17, a fixed mounting point is achieved for the hydro massage device 55. As shown in FIG. 17 the hydro massage device 55 is effectively mounted to the user and is made pivotal by virtue of the articulating nature of a ball and socket joint, or similar, at the base of attachment 43. Pre-determined on-center dimensions between the jet nozzle 12 and female socket 42, align the jet nozzle 12 to the upper labium and clitoral region 58 of the female user. The hydro massage device 55 can now be held and precisely aimed with very little effort. The added range of motion created by the articulating joint allows the handle to be moved in joystick fashion. This feature allows the user to control the distance of the jet to the target area and precisely adjust the trajectory of the jet to any particular point on the female user, such as the upper labium and clitoral region 58 for enhanced and controlled stimulation.

Furthermore, although not shown, it should be understood that the housing 1 could be formed with a distended portion located at the fore portion of the housing 1 so that it extends away from the housing 1. This distended portion could also be coated or covered with materials suitable for the application to allow insertion in an anal or a vaginal orifice of a human user's lower body to fix the position of the housing 1 in relation to the human user's lower body.

It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A bathing apparatus for a human user comprising:
a. A housing, wherein said housing has a rigid outer structure having a fore and an aft portion surrounding an internal cavity, said internal cavity for fluid flow through said rigid outer structure and wherein said housing has a plurality of exterior engagement surfaces for contact with a human user's lower body;
b. An air-water stream generator coupled to a water inlet orifice and an air inlet orifice, said air-water stream generator encompassed by said housing, wherein said water inlet orifice is in fluid communication with a water stream source and wherein said air inlet orifice is in fluid communication with an air source through an external air intake port connected to an air suction conduit;
c. A low voltage direct current pump means imparting an increase in pressure and flow of water within said housing to said outlet nozzle;
d. An electrical source electrically connected to said pump means;
e. A handle, wherein said handle is attached to said housing;
f. An air-water stream outlet nozzle located within said fore portion of said housing and connected to said air-water stream generator for transmission of said air-water stream onto said human user's lower body positioned fore of said air-water stream outlet nozzle;
g. A distended portion positioned at said fore portion of said housing wherein said distended portion extends away from said housing and upon insertion in an anal or a vaginal orifice in said human user's lower body fixes the position of said housing in relation to said human user's lower body; and,
h. Wherein said handle allows the human user to control said air-water stream outlet nozzle and stimulation upon said human user's genitals or genitalia by said air-water stream.

2. The bathing apparatus according to claim 1 and further wherein said distended portion is adjacent said air-water stream outlet nozzle.

3. The bathing apparatus according to claim 1 wherein said distended portion is an attachment which is detachable.

4. The bathing apparatus according to claim 3 and further wherein said housing has at least one socket formed therein for cooperative engagement with said attachment.

5. The bathing apparatus according to claim 4 and further wherein said at least one socket allows said attachment to articulate in relation to said housing.

6. The bathing apparatus according to claim 5 and further wherein an articulating joint is formed by the cooperative engagement of said at least one socket and said attachment.

7. The bathing apparatus according to claim 5 and further wherein said at least one socket is formed as the female portion of a ball and socket joint and said attachment has a male portion of ball and socket joint located at the base of said attachment.

8. The bathing apparatus according to claim 3 and further wherein said attachment is formed as a phallic extension with a g-spot massager.

9. The bathing apparatus according to claim 3 and further wherein said attachment is formed as a phallic extension with a prostrate massager.

10. The bathing apparatus according to claim 1 wherein said bathing apparatus is submerged in a water source and further wherein a bobber is fluidly attached to said external air intake port to maintain communication with said air source during submerged operation of said bathing apparatus.

11. The bathing apparatus according to claim 10 and further wherein said bobber is buoyant to float in said water source to maintain communication with said air source during submerged operation of said bathing apparatus in said water source.

12. The bathing apparatus according to claim 1 and further wherein said air-water stream outlet nozzle is an oscillating nozzle to produce an air-water stream having oscillating properties for transmission onto said human user's lower body for increased stimulation.

13. The bathing apparatus according to claim 12 and further wherein said oscillating nozzle is rotatable to allow said human user to control the direction of transmission of said air-water stream.

14. The bathing apparatus according to claim 12 and further wherein said air source is not in fluid communication with said air-water stream generator and said oscillating nozzle produces a water stream having oscillating properties for transmission onto said human user's lower body for increased stimulation.

15. The bathing apparatus according to claim 1 and further wherein said external air intake port is controllable by said user to produce an air-water stream having pulsation properties for transmission onto said human user's lower body for increased stimulation.

16. The bathing apparatus according to claim 10 and further wherein flow of air to said external air intake port is controllable by said bobber to produce an air-water stream having pulsation properties for transmission onto said human user's lower body for increased stimulation.

17. The bathing apparatus according to claim 16 further wherein said bobber is buoyant in said water source to maintain communication with said air source during submerged operation of said bathing apparatus in said water source.

18. The bathing apparatus according to claim 1 and further wherein said power source is a rechargeable battery located within said internal cavity.

19. The bathing apparatus according to claim 18 and further wherein said rechargeable battery is isolated within a second compartment within said internal cavity, said second compartment being waterproof.

20. The bathing apparatus according to claim 18 and further wherein said bathing apparatus may be electrically connected to a battery charger for recharging said rechargeable battery when said bathing apparatus is not operating and external of said water source and wherein said battery charger is electrically connected to a power source external of said bathing apparatus.

21. The bathing apparatus according to claim 18 and further wherein said bathing apparatus may be positioned upon an external battery charging structure for contact-less recharging of said rechargeable battery, wherein said external battery charging structure is electrically connected to an external power source.

22. The bathing apparatus according to claim 1 and further wherein said bathing apparatus has a chamfered portion on a side opposite said handle allowing said housing to pivot against a surface upon which said bathing apparatus may be positioned thereby allowing further control of the direction of transmission of said air-water stream.

23. A bathing apparatus for a female user comprising:
a. A housing wherein said housing is further comprised of a rigid outer structure having a fore and an aft portion surrounding an internal cavity, said internal cavity for fluid flow through said rigid outer structure and wherein said housing has a plurality of exterior engagement surfaces for contact with a female user's lower body;
b. A water inlet orifice positioned in said internal cavity and in fluid communication with a water stream source;
c. An air-water stream generator coupled to said water inlet orifice and said air inlet orifice encompassed by said rigid outer structure;
d. A handle, wherein said handle is attached to and continuous with said fore portion of said housing;
e. An air-water stream outlet nozzle located within said fore portion of said housing and connected to said water-air stream generator for transmission of said air-water stream onto said female user's lower body positioned fore of said air-water stream outlet nozzle;
f. An attachment positioned at and connected to said fore portion of said housing wherein said attachment extends away from said housing and upon insertion in said female user's vagina fixes the position of said housing in relation to said female user's lower body; and,
g. wherein said handle allows the female user to control said outlet nozzle and stimulation upon said female user's clitoris by said air-water stream.

24. The bathing apparatus according to claim 23 and further wherein said attachment is adjacent said outlet nozzle for simultaneous frictional stimulation of said female user's vagina.

25. The bathing apparatus according to claim 24 wherein said attachment is detachable.

26. The bathing apparatus according to claim 25 wherein said attachment is phallic shaped.

27. The bathing apparatus according to claim 23 and further wherein said housing has at least one socket formed therein for selective positioning of said attachment.

28. The bathing apparatus according to claim 27 and further wherein said at least one socket is formed as the female portion of a ball and socket joint.

29. A method of hydrotherapeutic stimulation of the female clitoral region in a contained water vessel using a hydro jet device compromising the following steps:
a. Positioning the user comfortably within the contained water vessel;
b. Placing the hydro jet device under the water between the user's legs in close proximity to said user's vagina;
c. Inserting a portion of the exterior of the hydro jet device in said user's vagina to fix the spatial relationship and position of the hydro jet device in relation to the user's vagina and clitoris;
d. Engaging the exterior of the hydro jet device and the user's body;
e. Accelerating and combining water and air in the hydro jet device to produce an air-water stream under pressure;
f. Discharging said air-water stream towards said user's clitoral region for clitoris stimulation; and,
g. Allowing the user to control the exterior of the hydro jet device through engagement with said user's lower body for vaginal stimulation while simultaneously allowing the user to control the discharge trajectory and intensity of said air-water stream during engagement and stimulation of said user's clitoral region.

30. The method of hydrotherapeutic stimulation of the female clitoral region according to claim 29 and further compromising maintaining a non-engagement space between the user's clitoral region and the exterior of the hydro jet device during stimulation of the clitoral region with said air-water mixture.

31. The method of hydrotherapeutic stimulation of the female clitoral region according to claim 29 wherein said portion of the exterior of the hydro jet device inserted in said user's vagina to fix the spatial relationship between the user and the hydro jet device is an attachment which is detachable from the hydro jet device.

32. The method of hydrotherapeutic stimulation of the female clitoral region according to claim 31 wherein said attachment is phallic shape.
